(19) United States
(12) Patent Application Publication
(10) Pub. No.: US 2025/0222240 A1
(43) Pub. Date: Jul. 10, 2025

(54) CATHETER AND SYSTEMS WITH FLEXIBLE BACKSTOP AND METHODS FOR FORMING FISTULAS

(71) Applicant: TVA MEDICAL GROUP, INC., Franklin Lakes, NJ (US)

(72) Inventors: Andrew Moll, Franklin Lakes, NJ (US); Oladipo Peter Akerle-ale, Franklin Lakes, NJ (US); Alexander Palmer, Franklin Lakes, NJ (US); Olivia Palmer, Franklin Lakes, NJ (US); Breanna Simpson, Franklin Lakes, NJ (US)

(21) Appl. No.: 18/853,370

(22) PCT Filed: Apr. 1, 2022
(86) PCT No.: PCT/US2022/023025

§ 371 Date: Oct. 1, 2024

(51) Int. Cl. A61M 27/00; A61B 18/00; A61B 18/14

(52) U.S. Cl. CPC A61M 27/002; A61B 18/1492; A61B 2018/00083; A61B 2018/00404; A61B 2018/00577; A61B 2018/00625; A61M 2205/0288

(57) ABSTRACT

Embodiments of systems, methods, and catheters for delivery of treatments to a blood vessel (e.g., fistula formation) using one or more catheters. Embodiments of the catheter or catheter system may include a first catheter including a first body, an electrode mounted to the first body, and a first array of magnets mounted to the first body, and a second catheter including a second body, a second array of magnets mounted to the second body, and a flexible backstop extending longitudinally along the second array of magnets. In embodiments, the presently disclosed catheters and catheter systems may provide endovascular fistula formation treatment that provide improved catheter flexibility.

### TECHNICAL FIELD

The present specification generally relates to systems, methods, and catheters for endovascular treatment of a blood vessel and, more specifically, systems, methods, and catheters for forming a fistula.

### BACKGROUND

Treatments such as endovascular fistula formation may include positioning two catheters within concomitant blood vessels to form a fistula therebetween. Forming a fistula between two blood vessels can have one or more beneficial functions. For example, the formation of a fistula between an artery and a vein may provide access to the vasculature for hemodialysis patients. Specifically, forming a fistula between an artery and a vein allows blood to flow quickly between the vessels while bypassing the capillaries. In other instances, a fistula may be formed between two veins to form a veno-venous fistula. It is often difficult to reach various locations within the vasculature of a subject due to its tortuous nature. The tortuous nature may also make it difficult to achieve desired coaptation between the two catheters to form a fistula.

Accordingly, a need exists for alternative systems, methods, and catheters that provide improved flexibility to traverse the vasculature of a subject to a desired location and/or coaptation between catheters for fistula formation.

### SUMMARY

The present embodiments address the above referenced problems. In particular, the present disclosure is directed to systems, methods, and catheters for fistula formation using catheters positioned in adjacent vessels. In embodiments, the presently-disclosed catheters and catheter systems may improve fistula formation procedures by providing, for example, improved catheter flexibility and coaptation.

In one embodiment, a system for forming a fistula between two vessels is provided. The system may include a first catheter including a first body, an electrode mounted to the first body, and a first array of magnets mounted to the first body. The system may include a second catheter including a second body, a second array of magnets mounted to the second body, and a flexible backstop extending longitudinally along the second array of magnets.

In another embodiment, a method of forming a fistula between two vessels is provided. The method may include advancing a first catheter into a first blood vessel, wherein the first catheter comprises a first treatment portion comprising an electrode and a first array of magnets, and advancing a second catheter into a second blood vessel. The second catheter may include a second body, a second array of magnets mounted to the second body, and a flexible backstop extending longitudinally along the second array of magnets. The method may include aligning the electrode with the flexible back stop, and ablating tissue with the electrode thereby forming a fistula between the first blood vessel and the second blood vessel.

In yet another embodiment, a catheter is provided. The catheter may include a body, an array of magnets mounted to the body, and a flexible backstop mounted to and extending longitudinally alongside the array of magnets, the flexible backstop defining a treatment region for receiving an electrode.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a system for forming a fistula between two vessels including a first catheter and a second catheter, according to one or more embodiments shown and described herein;

FIG. 2A schematically depicts a perspective view of a portion of the second catheter of FIG. 1, according to one or more embodiments shown and described herein.

FIG. 2B schematically depicts a cross sectional view of a portion of the second catheter of FIG. 1 taken along section line 2B-2B, according to one or more embodiments shown and described herein.

FIG. 3 schematically depicts a perspective view of another embodiment of a second catheter for use with the system of FIG. 1, according to one or more embodiments shown and described herein;

FIG. 4 depicts a flowchart illustrating a method for forming a fistula with the system of FIG. 1, according to one or more embodiments shown and described herein;

FIG. 5A schematically depicts the first catheter of FIG. 1 being advanced through a first blood vessel and the second catheter of FIG. 1 being advanced through a second vessel, according to one or more embodiments shown and described herein;

FIG. 5B schematically depicts alignment of the first catheter and the second catheter of FIG. 5A, according to one or more embodiments shown and described herein;

FIG. 5C schematically depicts treatment of the first blood vessel and the second blood vessel with the first catheter and the second catheter of FIG. 5B to form a fistula, according to one or more embodiments shown and described herein; and

FIG. 5D schematically depicts a fistula formed between the first blood vessel and the second blood vessel, according to one or more embodiments shown and described herein.

Reference will now be made in greater detail to various embodiments of the present disclosure, some embodiments of which are illustrated in the accompanying drawings. Whenever possible, the same reference numerals will be used throughout the drawings to refer to the same or similar parts.

### DETAILED DESCRIPTION

Embodiments as described herein are directed the systems, methods, and catheters for forming a fistula between two vessels. For example, fistulas may be formed between adjacent arteries and veins, adjacent veins, adjacent arteries, etc. In at least one embodiment, a system for forming a fistula generally includes a first catheter and a second catheter. The first catheter may include a first body, an electrode mounted to the first body, and a first array of magnets mounted to the first body. The second catheter may generally include a second body, a second array of magnets mounted to the second body, and a flexible backstop extending longitudinally along the second array of magnets. The flexible backstop facilitates flexure and/or bending of the second catheter along the flexible backstop, thereby allowing the second catheter to more easily traverse the tortuous vasculature of a subject. Moreover, the first and second arrays of magnets may also be flexible, thereby improving traversal of the vasculature. As will be described in greater detail, by having the flexible backstop overlie the second array of magnets, magnetic elements may extend through a treatment portion of the catheter, thereby providing a larger and/or stronger coaption region between the first and second catheter. These and additional embodiments and benefits will be described in greater detail herein.

As used herein, the term “flexible” generally refers to the ability of an object to be bent in one or more directions without breaking. Degrees of flexibility may vary, however, flexible objects may be able to bend from a substantially linear orientation to a substantially curved or angled orientation, such as between about 0° and about 180°, such as between about 10° and about 170°, such as between about 20° and about 160°, such as between about 40° and about 140°, etc. Accordingly, embodiments including flexible elements as described herein, easily flex to traverse a tortuous anatomy of a subject.

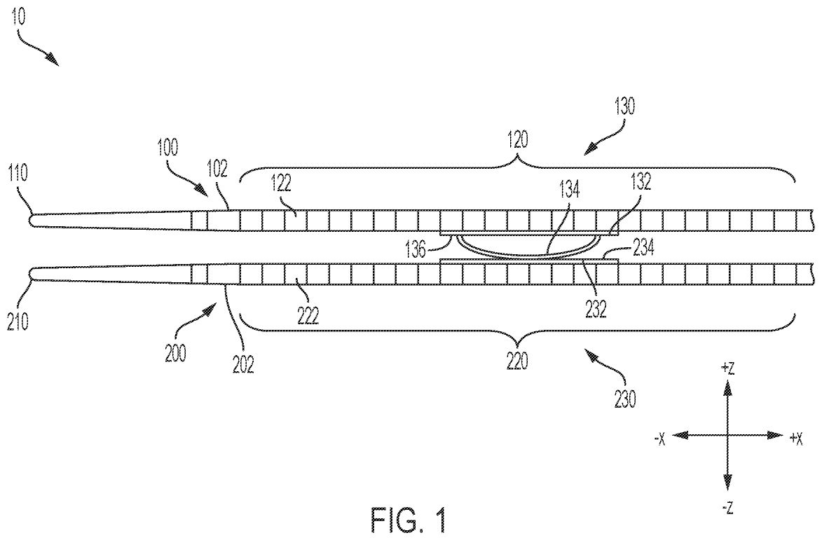

Referring now to FIG. 1, a system 10 for forming a fistula between two vessels is generally depicted. The system 10 may generally include a first catheter 100 and a second catheter 200. It may be appreciated that the first catheter 100 and the second catheter 200 are substantially similar to one another. Accordingly, description of the first catheter 100 generally applies to the second catheter 200 unless otherwise noted or apparent. It is noted that the first catheter 100 and the second catheter 200 may be provided within a kit and/or separately from one another.

The first catheter 100 generally includes a catheter body 102, a treatment portion 130, and a magnetic array 120. It is noted that the first catheter 100 may include a greater or fewer number of components without departing from the scope of the present disclosure.

The catheter body 102 may be sized to be advanced through a blood vessel and may include a distal tip 110 that may be shaped and/or sized to aid in advancement of the first catheter 100 through a blood vessel. For example, the distal tip 110 may be pointed, tapered, and/or atraumatic for advancement through a blood vessel. The catheter body 102 may have any cross-sectional shape and any diameter suitable for intravascular use. The first catheter 100 may include or define one or more lumens or other passageways (not shown) extending at least partially along or through the catheter body 102. For instance, the one or more lumens may extend at least partially longitudinally through the catheter body 102 along an x-axis of the depicted coordinate axes of FIG. 1. The catheter body 102 may be formed of any material or combination of materials able to be traversed through a vasculature of a body. For example, the catheter body 102 may include, silicone, rubber, Pebax (i.e., polyether block amide), paralene, etc.

As noted above, the first catheter 100 may have a treatment portion 130 for forming a fistula. In embodiments, the treatment portion 130 may have a fistula-forming element 134 that is disposed in a housing 132, coupled to or part of the catheter body 102. In embodiments, the fistula-forming element 134 may be an electrode, such as a leaf spring electrode, having an exposed ablation surface. The fistula-forming element 134 may be coupled to a power source (not shown), such as via a lead wire or other conductor attached thereto. When activated, current may be supplied to and/or carried from tissue and fluid via the ablation surface to facilitate ablation or vaporization of tissue to form a fistula.

In some embodiments, the fistula-forming element 134 may be a spring wire or leaf spring electrode, which may be movable between a retracted configuration, in which the fistula-forming element 134 is retained within the first catheter 100, such as the housing 132, and a protruding configuration, in which the fistula-forming element 134 projects beyond the outer surface 136 of housing 132. For example, the housing 132 may define a lateral opening through which the fistula-forming element 134 may extend or retract. The fistula-forming element 134 may or may not be naturally biased to project from the catheter body 102. When the fistula-forming element 134 is naturally biased to project from the catheter body 102, a structure, such as a sleeve (not shown), may be used to hold or maintain the fistula-forming element 134 in a retracted configuration until deployment is desired. In some embodiments, the fistula-forming element 134 may be manually advanced or retracted such as via a lead wire which may be coupled to the fistula-forming element 134 which may be manually pulled or pushed to retract or advance the fistula-forming element 134. In some embodiments, the first catheter 100 may comprise one or more insulating materials (not shown) which may shield or otherwise protect the first catheter 100 and its components from heat generated by the fistula-forming element 134 during use.

As depicted, the fistula-forming element 134 may be arc shaped, though other shapes are contemplated and possible (e.g., rectangular, square, angular, etc.). The size and shape of the fistula-forming element 134 may be varied based on factors including tissue thickness and density, as well as desired fistula size, shape, and location. It is noted that the fistula-forming element 134 is not limited to an electrode as describe above, but may include a different cutting/ablation device such as, but not limited to, any electrocautery mechanism, blades, lances, needles, cryogenic-cautery devices, ultrasonic-cautery devices, laser ablation devices, etc.

Still referring to FIG. 1 and as noted above, the first catheter 100 may have a magnetic array 120 arranged on or within the catheter body 102. The magnetic array 120 may include a plurality of magnetic elements 122 arranged in a longitudinal array along a length of the catheter. In some embodiments, the magnetic array 120 extends longitudinally along the treatment portion 130 of the first catheter 100 so as to be overlie the treatment portion 130 in the +Z direction of the depicted coordinate axes. For example, the plurality of magnetic elements 122 may be disposed along the catheter body 102 of the first catheter 100. It is noted that magnetic elements overlying the treatment portion 130 of the first catheter 100 may have smaller dimension (e.g., size) than magnetic elements positioned to either side of the treatment portion 130. Accordingly, the side of the catheter through the treatment portion may be generally kept to the profile size of adjacent portions.

Generally, the dimensions of the magnets described herein may be selected based on the size of the catheters carrying the magnets, which in turn may be selected based on the anatomical dimensions of the blood vessels through which the catheters may be advanced. For example, if the catheter is to be advanced through a blood vessel having an internal diameter of about 3 mm, it may be desirable to configure any magnet to be less than about 3 mm at the widest part of its cross-section, to reduce the risk of injury to vessel walls during advancement and manipulation of the catheter. Each magnet may have any suitable length (e.g., about 5 mm, about 10 mm, about 15 mm, about 20 mm, or the like). Although, it should be appreciated that in some instances longer magnets may limit the flexibility of the catheter to maneuver through tissue. For example, the magnetic elements 122 of the magnetic array 120 may connected to each other such that the connection between the magnetic elements form joints to flex or bend the first catheter 100. For example, the magnetic elements of the magnetic array 120 may be arranged within the catheter body 102, such that the catheter body generally holds the magnetic elements of the magnetic array together while allowing the magnetic array to flex and/or undulate to traverse the vasculature of a subject. Accordingly, the magnetic array 120 may be flexible.

In some embodiments, the number of the plurality of magnetic elements 122 of the magnetic array 120 may be modified for optimization of magnetic strength for alignment or coaptation purposes. The magnetic array 120 may comprise any number of individual magnetic elements, such as 10 or more magnetic elements, 20 or more magnetic elements, 30 or more magnetic elements, 60 or more magnetic elements, etc. The magnetic array 120 may be continuous or may be broken into a plurality of magnetic arrays, such as two or more magnetic arrays, three or more magnetic arrays, etc.

The magnetic elements 122 may include permanent magnets comprising one or more hard magnetic materials, such as but not limited to alloys of rare earth elements (e.g., samarium-cobalt magnets or neodymium magnets, such as N52 magnets) or alnico. In some variations, the magnetic elements 122 may comprise anisotropic magnets; in other variations, the magnetic elements may comprise isotropic magnetics. In some variations, the magnetic elements 122 may be formed from compressed powder. In some variations, the magnetic elements 122 may include one or more soft magnetic materials, such as but not limited to iron, cobalt, nickel, or ferrite.

Referring also to FIG. 1, similar to the first catheter 100, the second catheter 200 generally includes a catheter body 202, a treatment portion 230 including a backstop 234, and a magnetic array 220. It is noted that the second catheter 200 may include a greater or fewer number of components without departing from the scope of the present disclosure.

The catheter body 202 may be sized to be advanced through a blood vessel and may include a distal tip 210 that is may be shaped and/or sized to aid in advancement of the second catheter 200 through a blood vessel. For example, the distal tip 210 may be pointed, tapered, and/or atraumatic for advancement through a blood vessel. The catheter body 202 may have any cross-sectional shape and any diameter suitable for intravascular use. The second catheter 200 may include or define one or more lumens or other passageways (not shown) extending at least partially along or through the catheter body 202. For instance, the one or more lumens may extend at least partially longitudinally through the catheter body 202 in the direction of an X-axis of the depicted coordinate axes of FIG. 1. The catheter body 202 may be formed of any material or combination of materials able to be traversed through a vasculature of a body. For example, the catheter body 202 may include, silicone, rubber, Pebax, paralene, etc.

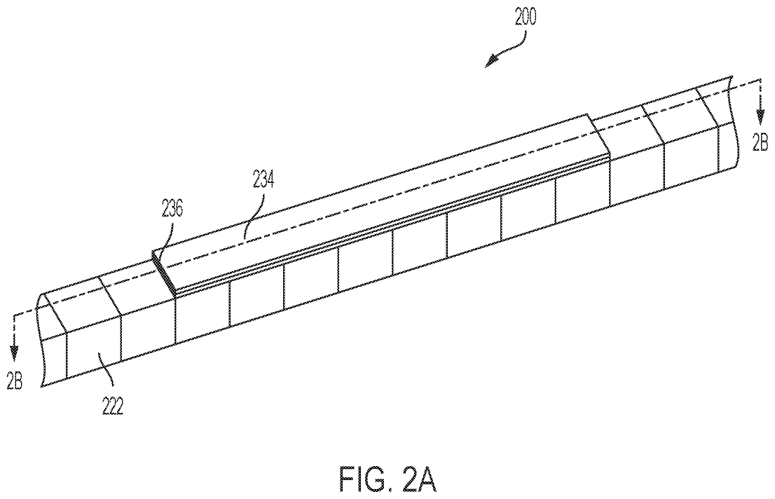

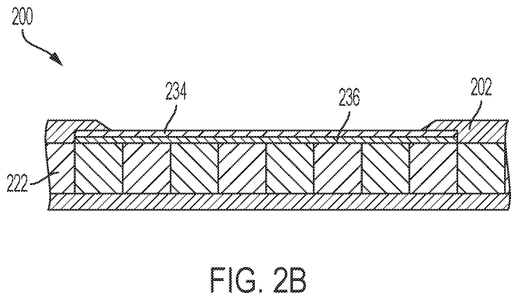

As noted above, the second catheter 200 may have a treatment portion 230 that may be aligned or coapted with the treatment portion 130 of the first catheter 100 such as when the first catheter 100 and the second catheter 200 are aligned in adjacent vessels. The treatment portion 230 may include a backstop 234 overlying the magnetic array 220, which will be discussed later in detail with reference to FIGS. 2A and 2B. The backstop 234 overlie the magnetic array 220 may provide a larger and/or stronger coaption region between the first catheter 100 and the second catheter 200 because the backstop 234 is flexible to be bend along the second catheter. The flexibility of the backstop 234 helps the second catheter to be bend along with a blood vessel and/or the first catheter 100.

The backstop 234 may be configured to receive the fistula-forming element 134 as it passes through tissue of the adjacent vessels to form a fistula. In some embodiments, the backstop 234 is made of a flexible material. The flexibility of the backstop 234 may be configured to conform to a shape of the second catheter 200 when acted upon by a bending force. The bending force may be from the second catheter 200 being bent while traversing tortuous anatomy of a vessel, thereby conforming the backstop 234 to the shape of the vessel. In addition, the bending force may be from the first catheter 100 and/or the fistula-forming element 134 being pressed onto the backstop 234 when the first catheter 100 and the second catheter 200 are aligned and coapted.

For example, the backstop 234 may be formed from a ribbon (e.g., a rolled wire) made from a metal material, such as but not limited to, tungsten rhenium, stainless steel, titanium, etc. In some embodiments, the backstop 234 may be magnetic, formed of a low corrosive metal material (e.g., ferrous or non-ferrous). As depicted the backstop 234 may be a thin ribbon overlying (e.g., in the +Z direction of the depicted coordinated axes) the magnetic array 220. For example, the backstop 234 may have a thickness in the +/−Z direction of the depicted coordinate axes of about 0.05 mm or less, such as about 0.02 mm or less, though thicker or slimmer thicknesses are contemplated and possible. The thickness may be from about 0.01 mm to about 0.03 mm. Accordingly, due to the thin nature of the backstop 234 in the +/−Z direction of the depicted coordinate axes, the backstop 234 may be flexible (thereby providing a flexible backstop) to provide enhanced traversal of a blood vessel by bending able to bend along with the magnetic array 220 as the second catheter 200 traverses a vessel. That is, the flexibility of the backstop 234 may help advancing the second catheter 200 through an anatomy.

The backstop 234 may be coupled to the magnetic array 220 via any suitable means. For example, the backstop 234 may be coupled to the magnetic array 220 via the catheter body 202. For example, the backstop 234 may be fixed to the catheter body 202 to hold the backstop 234 in place over the magnetic array. In some, embodiments the backstop 234 may be adhered to the magnetic array 220 via an adhesive layer. The backstop 234 may extend along any portion or length of the catheter body 202. For example, in some embodiments, the backstop 234 may extend along an entire length of the magnetic array 220. In some embodiments, the length of the backstop 234 may be equal to a length of the fistula forming element. In some embodiments, there may be multiple backstops 234 arranged along the magnetic array 220. In some embodiments, there may be a backstop 234 arranged on different surfaces of the magnetic array. For example, in the illustrated embodiment, the magnetic array include four planar surfaces (though a greater or fewer number of planar surfaces are contemplated and possible). Accordingly, in some embodiments, a backstop 234 may be arranged along each planar surface of the magnetic array. It is noted that while the backstop 234 is illustrated as having a rectangular shaped surface, other shapes are contemplated and possible. For example, edges of the backstop 234 may have any combination of curves, angles, or the like. In some embodiments, the backstop 234 may have a varying thickness (e.g., in the +/−Y direction and/or the +/−Z direction of the depicted coordinate axes.

Still referring to FIG. 1 and as noted above, the second catheter 200 may have a magnetic array 220 arranged on or within the catheter body 202. The magnetic array 220 may include a plurality of magnetic elements 222 arranged in a longitudinal array along a length of the catheter. In some embodiments, the magnetic array 220 extends along the treatment portion 230 of the second catheter 200 so as to be underlie the treatment portion 230 in the −Z direction of the Z direction of the depicted coordinate axes. For example, the magnetic elements of the magnetic array 220 may be arranged within the catheter body 202, such that the catheter body 202 generally holds the magnetic elements of the magnetic array together while allowing the magnetic array to flex and/or undulate to traverse the vasculature of a subject. It is noted that magnetic elements underlying the treatment portion 230 of the second catheter 200 may have smaller dimension (e.g., size) than magnetic elements positioned to either side of the treatment portion 230. Accordingly, the side of the catheter through the treatment portion 230 may be generally kept to the profile size of adjacent portions.

Generally, the dimensions of the magnets described herein may be selected based on the size of the catheters carrying the magnets, which in turn may be selected based on the anatomical dimensions of the blood vessels through which the catheters may be advanced. For example, if the catheter is to be advanced through a blood vessel having an internal diameter of about 3 mm, it may be desirable to configure any magnet to be less than about 3 mm at the widest part of its cross-section, to reduce the risk of injury to vessel walls during advancement and manipulation of the catheter. Each magnet may have any suitable length (e.g., about 5 mm, about 10 mm, about 15 mm, about 20 mm, or the like). Although, it should be appreciated that in some instances longer magnets may limit the flexibility of the catheter to maneuver through tissue. For example, the magnetic elements 222 of the magnetic array 220 may connected to each other such that the connection between the magnetic elements to form joints to flex or bend the second catheter 200. Accordingly, the magnetic array 220 may be flexible.

In some embodiments, the number of the plurality of magnetic elements 222 of the magnetic array 220 may be modified for optimization of magnetic strength for alignment or coaptation purposes. The magnetic array 220 may comprise any number of individual magnetic elements, such as 10 or more magnetic elements, 20 or more magnetic elements, 30 or more magnetic element, 60 or more magnetic elements, etc. The magnetic array 220 may be continuous or may be broken into a plurality of magnetic arrays, such as two or more magnetic arrays, three or more magnetic arrays, etc. The size of magnetic elements 122 that are disposed under the housing 132 and/or the magnetic elements 222 disposed under the backstop 234 may be smaller than the rest of the magnetic elements.

The magnets described here throughout may be permanent magnets comprising one or more hard magnetic materials, such as but not limited to alloys of rare earth elements (e.g., samarium-cobalt magnets or neodymium magnets, such as N52 magnets) or alnico. In some variations, the magnetic elements 222 may comprise anisotropic magnets; in other variations, the magnets may comprise isotropic magnetics. In some variations, the magnetic elements 222 may be formed from compressed powder. In some variations, the magnetic elements 222 may include one or more soft magnetic materials, such as but not limited to iron, cobalt, nickel, or ferrite.

In some embodiments, referring to FIG. 2A, the backstop 234 may be shaped as a strip and extend along the lateral direction of the second catheter 200. The backstop 234 may be made of a flexible material such as a ribbon (e.g., a rolled wire) or a metal strip. Material of the backstop 234 may be, but not limited to, tungsten rhenium, stainless steel, titanium, etc. In some embodiments, the backstop 234 may be magnetic, formed of a low corrosive metal material (e.g., ferrous or non-ferrous). As depicted, the backstop 234 may be a thin ribbon overlying the magnetic array 220. The backstop 234 may be exposed through a portion of a catheter body as will be shown in FIG. 2B.

In some embodiments, referring to FIG. 2B is a cross sectional view of the second catheter 200 of FIG. 2A taken along section line 2B-2B. Although not shown in FIG. 2A, the catheter body 202 may extend over and/or partially cover one or more portions of the magnetic array 220 and the backstop 234. In some embodiments, a portion of the catheter body 202 is arranged (or reflowed) to overlie the backstop 234. The portion of the catheter body 202 may be etched, carved, or otherwise removed to expose a portion of the backstop 234 configured to receive the fistula-forming element 134. For example, end portions of the backstop 234 in the lateral direction may be disposed under or covered by the catheter body 202. In some embodiments, the second catheter and/or the catheter body may include an insulative layer 236 disposed between the backstop 234 and the magnetic elements 222. For example, the insulative layer 236 may comprise one or more insulative materials and/or coatings to electrically and/or thermally insulate one or more components of the second catheter 200 during fistula formation (e.g., such as where the fistula-forming element 134 is an electrode). The insulative layer 236 may include one or more insulative materials that may insulate the backstop 234 from other metallic components (e.g., magnets). The insulative materials may include silicone, parylene, rubber, Pebax (i.e., polyether block amide), etc. Such insulative materials may be provided in a thickness (e.g., in the +/−Z direction of the depicted coordinate axes) such as about 0.5 mm or less, such as about 0.2 mm or less, though thicker or slimmer thicknesses are contemplated and possible. In some embodiments, the top surface of the backstop 234 may be substantially flush with the top surface of the catheter body 202. The top surface of the backstop 234 may be lower or higher than the top surface of the catheter body 202 such that the backstop 234 is configured to receive the fistula-forming element 134.

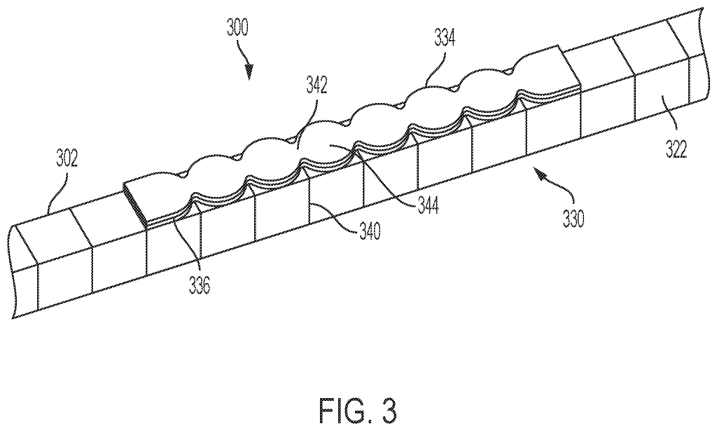

FIG. 3 illustrates an alternative embodiment of a backstop 334 of a catheter, which may be used in the second catheter of FIGS. 1, 2A, and 2B. It is noted that the above description applies equally to the present embodiment, unless otherwise noted. In particular, referring to FIG. 3, the backstop 334 may have a varying width along a length of the backstop 334. For example, the backstop 334 may have one or more narrow portions 342 and one or more wide portions 344 that are wider than the one or more narrow portions 342 in the +/−Y direction (e.g., lateral direction) of the depicted coordinate axes. In the depicted embodiment, each magnetic element 322 may sit adjacent another magnetic element such that an interface 340 is positioned between each adjacent magnetic element. The narrow portions 342 may be aligned to be overlapped with the interface 340 between the adjacent magnetic elements 322. By aligning the narrow portions 342 to the interfaces 340 between the adjacent magnetic elements 322, flexibility may be further improved as there is less material to restrict movement at the interfaces. In some embodiments, the catheter and/or the catheter body may include an insulative layer 336 disposed between the backstop 334 and the magnetic elements 322. The insulative layer 336 may be shaped substantially identical to the backstop 334 such that the insulative layer 336 may also have one or more narrow portions and one or more wide portions.

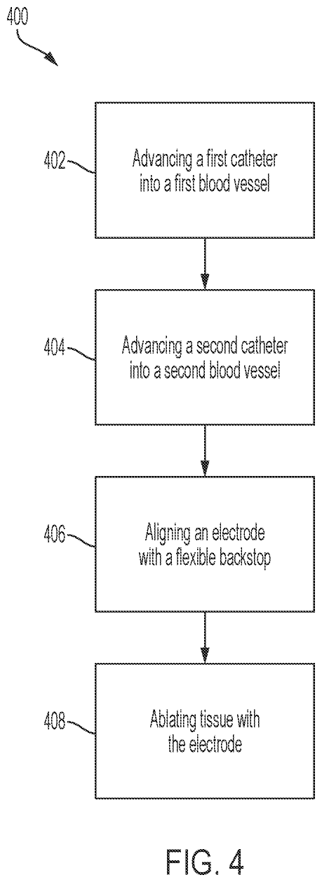

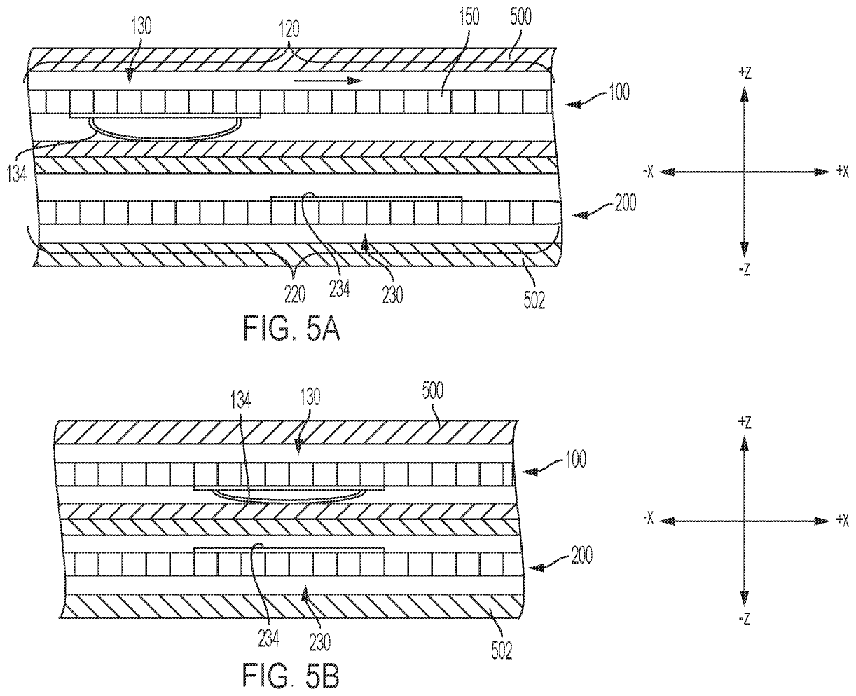

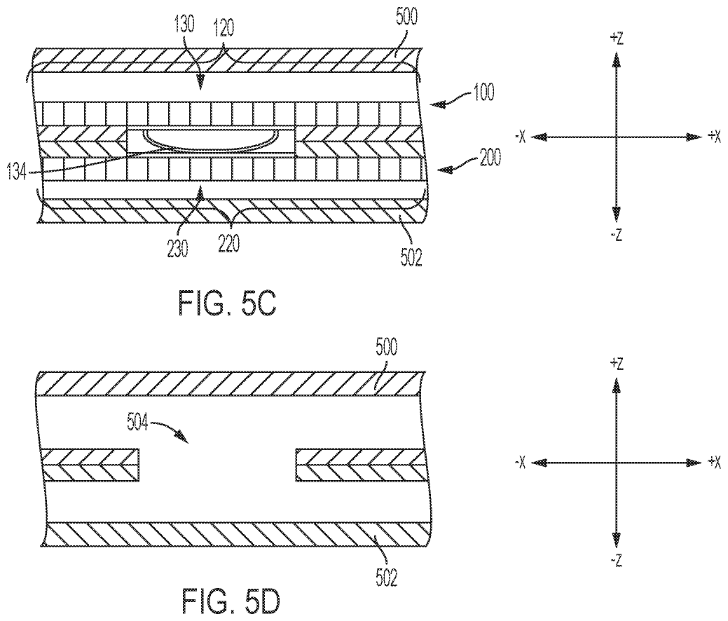

Referring to FIG. 4, a flow chart illustrating a method 400 of forming a fistula is generally depicted. It is noted that the method 400 may include a greater or fewer number of steps, taken in any order, without departing from the scope of the present disclosure. It is noted that the method 400 illustrated in FIG. 4 may be best understood when reviewed in conjunction with FIGS. 5A-5D which generally illustrate alignment and treatment of a blood vessel using the first and second catheters 100, 200 as described herein.

At block 402, the method 400 includes advancing the first catheter 100 through a first blood vessel 500 as depicted in FIG. 5A. For example, a user may advance the first catheter 100 through the first blood vessel 500 to a first treatment location so that the treatment portion 130 is advanced to the first treatment location.

Referring again to FIG. 4, at block 404 the method 400 further includes advancing the second catheter 200 through a second blood vessel 502. For example, and as depicted in FIG. 5A, the user may advance the second catheter 200 through the second blood vessel 502 to a second treatment location in the second blood vessel 502. It is noted that the first blood vessel 500 and the second blood vessel 502 may be adjacent vessels such as a vein and an artery, though vein to vein and artery to artery treatments are contemplated and possible.

Referring again to FIG. 4, at block 406 the method 400 includes aligning the treatment portion 130 of the first catheter 100 with the treatment portion 230 of the second catheter 200, as depicted in FIG. 5B. For example, as depicted in FIGS. 5B and 5C, once the treatment portions 130, 230 of the first catheter 100 and the second catheter 200 are aligned (e.g., both longitudinally and rotationally) the first and second catheters 100 may become attracted to one another via attraction between the magnetic array 120 of the first catheter 100 and the magnetic array 220 of the second catheter 200. Thus, the fistula-forming element 134 may be aligned with the backstop 234. A portion of the magnetic array 220 may be positioned beneath the backstop 234, and a portion of the magnetic array 120 may be positioned beneath the fistula-forming element 134. The attraction between the first and second catheters 100, 200 may cause the first catheter 100 and the second catheter 200 to snap together, thereby pulling the first blood vessel 500 and the second blood vessel 502 into contact with one another, as illustrated in FIG. 5C. Accordingly, once aligned, the first catheter 100 and the second catheter 200 may coapt together and sandwich or compress tissue of the first vessel and the second vessel therebetween. The backstop 234 may therefore define a compression region configured to receive the fistula-forming element 134 such that tissue is compressed between the backstop 234 and the fistula-forming element 134. In some embodiments, the method 300 may further include confirming alignment of the first catheter 100 and the second catheter 200 via fluoroscopy or other imaging techniques.

Still referring to FIG. 4, at block 408 the method 400 may further include, after alignment between the treatment portions 130, 230 of the first catheter 100 and the second catheter 200, performing a vascular treatment procedure to modify the first blood vessel 500 and/or the second blood vessel 502 at the first treatment location and/or the second treatment location. For example, the fistula-forming element 134 of the treatment portion 130 may be activated or energized (e.g., via an energy delivery device) to ablate tissue of the first blood vessel 500 and the second blood to form a fistula 504 between the first blood vessel 500 and the second blood vessel 502, as generally depicted in FIG. 5D. Upon formation of the fistula 504 the first catheter 100 and the second catheter 200 may be withdrawn from the respective first blood vessel 500 and the second blood vessel 502.

Embodiments may be further described with reference to the following numerical clauses:

1. A system for forming a fistula between two vessels comprising: a first catheter comprising a first body, an electrode mounted to the first body, and a first array of magnets mounted to the first body; and a second catheter comprising a second body, a second array of magnets mounted to the second body, and a flexible backstop extending longitudinally along the second array of magnets.

2. The system of any preceding clause, wherein a portion of the first array of magnets extends along the electrode in longitudinal direction of the first catheter.

3. The system of any preceding clause, wherein the flexible backstop comprises a metallic sheet.

4. The system of any preceding clause, wherein the flexible backstop defines a compression region configured to receive the electrode such that tissue is compressed between the flexible backstop and the electrode when the first and second catheters are positioned such that the first and second array of magnets attract each other.

5. The system of any preceding clause, wherein the second catheter further comprises an insulative layer disposed between the flexible backstop and the second array of magnets.

6. The system of any preceding clause, wherein the first array of magnets is configured to conform to a shape of the first catheter when acted upon by a bending force and/or the second array of magnets is configured to conform to a shape of the second catheter when acted upon by a bending force.

7. The system of any preceding clause, wherein the flexible backstop is configured to conform to a shape of the second catheter when acted upon by a bending force.

8. The system of any preceding clause, wherein the flexible backstop has a rectangular shaped surface.

9. The system of any preceding clause, wherein the flexible backstop comprises a varying width along a length of the flexible backstop, the varying width comprising one or more narrow portions and one or more wide portions which are wider than the one or more narrow portions in a lateral direction.

10. The system of any preceding clause, wherein the one or more narrow portions of the flexible backstop are aligned with one or more magnet interfaces between individual magnets in the second array of magnets.

11. A method of forming a fistula between two vessels comprising: advancing a first catheter into a first blood vessel, wherein the first catheter comprises a first treatment portion comprising an electrode and a first array of magnets; advancing a second catheter into a second blood vessel, the second catheter comprising a second body, a second array of magnets mounted to the second body, and a flexible backstop extending longitudinally along the second array of magnets; aligning the electrode with the flexible backstop; and ablating tissue with the electrode thereby forming a fistula between the first blood vessel and the second blood vessel.

12. The method of any preceding clause, wherein the flexible backstop is configured to conform to a shape of the second catheter when acted upon by a bending force.

13. The method of any preceding clause, wherein magnetic attraction forces between the first array of magnets and the second array of magnets compress tissue between the flexible backstop and the electrode at the compression region.

14. A catheter comprising: a body; an array of magnets mounted to the body; and a flexible backstop mounted to and extending longitudinally alongside the array of magnets, the flexible backstop defining a treatment region for receiving an electrode.

15. The catheter of any preceding clause, wherein the array of magnets is configured to conform to a shape of the catheter when acted upon by a bending force.

16. The catheter of any preceding clause, wherein the flexible backstop comprises a metallic sheet.

17. The catheter of any preceding clause, wherein the body extends over a portion of the metallic sheet and defines an opening exposing the treatment region.

18. The catheter of any preceding clause, wherein the flexible backstop is configured to conform to a shape of the second catheter when acted upon by a bending force.

19. The catheter of any preceding clause, wherein the metallic sheet has a rectangular shaped surface.

20. The catheter of any preceding clause, wherein: the metallic sheet comprises a varying width along a length of the flexible backstop, the varying width comprising one or more narrow portions and one or more wide portions which are wider than the one or more narrow portions in a lateral direction; and the one or more narrow portions of the metallic sheet are aligned with one or more magnet interfaces between individual magnets in the array of magnets.

It should now be understood that embodiments of the present disclosure are directed to devices, systems, and methods for forming a fistula between two blood vessels. In some embodiments, the devices and methods may be used to form a fistula between two blood vessels. More particularly, a catheter may be placed in each of two adjacent blood vessels to form a fistula therebetween with the catheters. For example, in some embodiments, a catheter for endovascular treatment of a blood vessel includes a treatment portion comprising a metallic backstop, and a magnetic array. The metallic backstop may improve fistula formation, increase flexibility, and enhance coaption.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A system for forming a fistula between two vessels comprising:
a first catheter comprising a first body, an electrode mounted to the first body, and a first array of magnets mounted to the first body; and
a second catheter comprising a second body, a second array of magnets mounted to the second body, and a flexible backstop extending longitudinally along the second array of magnets.

2. The system of claim 1, wherein a portion of the first array of magnets extends along the electrode in longitudinal direction of the first catheter.

3. The system of claim 1, wherein the flexible backstop comprises a metallic sheet.

4. The system of claim 1, wherein the flexible backstop defines a compression region configured to receive the electrode such that tissue is compressed between the flexible backstop and the electrode when the first and second catheters are positioned such that the first and second array of magnets attract each other.

5. The system of claim 4, wherein the second catheter further comprises an insulative layer disposed between the flexible backstop and the second array of magnets.

6. The system of claim 1, wherein the first array of magnets is configured to conform to a shape of the first catheter when acted upon by a bending force and/or the second array of magnets is configured to conform to a shape of the second catheter when acted upon by a bending force.

7. The system of claim 1, wherein the flexible backstop is configured to conform to a shape of the second catheter when acted upon by a bending force.

8. The system of claim 1, wherein the flexible backstop has a rectangular shaped surface.

9. The system of claim 1, wherein the flexible backstop comprises a varying width along a length of the flexible backstop, the varying width comprising one or more narrow portions and one or more wide portions which are wider than the one or more narrow portions in a lateral direction.

10. The system of claim 9, wherein the one or more narrow portions of the flexible backstop are aligned with one or more magnet interfaces between individual magnets in the second array of magnets.

11. A method of forming a fistula between two vessels comprising:
advancing a first catheter into a first blood vessel, wherein the first catheter comprises a first treatment portion comprising an electrode and a first array of magnets;
advancing a second catheter into a second blood vessel, the second catheter comprising a second body, a second array of magnets mounted to the second body, and a flexible backstop extending longitudinally along the second array of magnets;
aligning the electrode with the flexible backstop; and
ablating tissue with the electrode thereby forming a fistula between the first blood vessel and the second blood vessel.

12. The method of claim 11, wherein the flexible backstop is configured to conform to a shape of the second catheter when acted upon by a bending force.

13. The method of claim 12, wherein magnetic attraction forces between the first array of magnets and the second array of magnets compress tissue between the flexible backstop and the electrode at the compression region.

14. A catheter comprising:
a body;
an array of magnets mounted to the body; and
a flexible backstop mounted to and extending longitudinally alongside the array of magnets, the flexible backstop defining a treatment region for receiving an electrode.

15. The catheter of claim 14, wherein the array of magnets is configured to conform to a shape of the catheter when acted upon by a bending force.

16. The catheter of claim 14, wherein the flexible backstop comprises a metallic sheet.

17. The catheter of claim 16, wherein the body extends over a portion of the metallic sheet and defines an opening exposing the treatment region.

18. The catheter of claim 16, wherein the flexible backstop is configured to conform to a shape of the catheter when acted upon by a bending force.

19. The catheter of claim 16, wherein the metallic sheet has a rectangular shaped surface.

20. The catheter of claim 16, wherein:
the metallic sheet comprises a varying width along a length of the flexible backstop, the varying width comprising one or more narrow portions and one or more wide portions which are wider than the one or more narrow portions in a lateral direction; and
the one or more narrow portions of the metallic sheet are aligned with one or more magnet interfaces between individual magnets in the array of magnets.
